(19) United States
(12) Patent Application Publication
(10) Pub. No.: US 2026/0159470 A1
(43) Pub. Date: Jun. 11, 2026

(54) PROCESS FOR RECOVERING ACRYLIC ACID VALUE FROM HEAVY WASTE BYPRODUCTS

(71) Applicants: Dow Global Technologies LLC, Midland, MI (US); Rohm and Haas Company, Collegeville, PA (US)

(72) Inventors: Vipul Sharma, King of Prussia, PA (US); Kirk W. Limbach, Dresher, PA (US); Thomas Goodall, Houston, TX (US); Daniel J. Martenak, Perkasie, PA (US); Reetam Chakrabarti, Phoenixville, PA (US); Joseph F. Dewilde, King of Prussia, PA (US); Nelson I. Quiros, Houston, TX (US)

(21) Appl. No.: 19/391,364

(22) Filed: Nov. 17, 2025

Related U.S. Application Data
(60) Provisional application No. 63/728,481, filed on Dec. 5, 2024.

(51) Int. Cl. C07C 51/44

(52) U.S. Cl. CPC C07C 51/44

(57) ABSTRACT

A process for producing acrylic acid and recovering acrylic acid from Michael addition products comprises feeding a crude acrylic acid stream to a dehydration tower to produce an overhead stream comprising noncondensibles and lights and a bottoms stream comprising acrylic acid and Michael addition products of acrylic acid. At least a portion of the bottoms stream from the dehydration tower is fed to a finishing tower to distill the bottoms stream of the dehydration tower to produce a side draw stream comprising acrylic acid, and a bottoms stream comprising acrylic acid and Michael addition products of acrylic acid. Optionally, acrylic acid from the acrylic acid-rich stream is fed to an esterification process and/or a purification process. The esterification process produces an acrylic acid ester from the acrylic acid-rich stream, wherein a waste stream from the esterification process comprises Michael addition products of acrylic acid. The purification process produces a purified acrylic acid stream from the acrylic acid-rich stream, wherein a waste stream from the purification process comprises Michael addition products of acrylic acid. At least a portion of the bottoms stream from the finishing tower and/or the waste stream from the esterification process and/or the purification process is fed to a distillation column. A distillate comprising acrylic acid and a bottoms stream comprising the Michael addition products of acrylic acid are removed from the distillation column. At least a portion of the bottom stream from the distillation column is fed to a dimer cracker reactor. Acrylic acid is recovered from a vapor stream exiting the dimer cracker reactor.

### FIELD OF THE INVENTION

The invention relates to a method for recovering acrylic acid value from heavy waste byproducts.

### BACKGROUND OF THE INVENTION

Acrylic acid and its ester derivatives are used in the manufacture of a broad spectrum of polymer products found across multiple market segments. For example, poly-acrylic acid (PAA) formulations are commonly found in detergent applications, sodium salts of PAA are used as super absorbent polymers (SAP) found in baby diapers and other adult hygiene products. Copolymers of acrylic monomers with methacrylic monomers, styrene, butadiene and other functionalized vinyl monomers are used to manufacture dispersions and resins that are ubiquitous in consumer and industrial products such as architectural, industrial, paper and automotive coatings, performance additives for concrete admixtures and asphalts, textiles, adhesives, sealants and caulks to name just a few applications.

Commercially, acrylic monomer production is dominated by acrylic acid which serves as a platform chemical for a broad range of acrylate derivatives. Acrylic acid may be produced using a variety of different methods. For example, acrylic acid can be made via propylene oxidation, bio-based processes (e.g., using any one of glycerol, 3-hydroxypropionic acid, lactate, lactic acid, etc., as a starting material), the ethylene cyanohydrin process, the acetylene process (i.e., the Reppe process), the β-propiolactone process, acrylonitrile hydrolysis, or coupling carbon dioxide with ethylene or ethane. Industrially, most of the global capacity of acrylic acid is manufactured via the two-stage heterogeneously catalyzed oxidation of propylene over mixed metal oxide catalysts.

The crude acrylic acid (CAA) that is produced by any of the known methods contains Michael addition products, which include dimers, trimers, and higher-mers of acrylic acid. These Michael addition products may reversibly react to form acrylic acid or higher or lower oligomers.

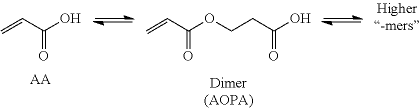

Michael addition products of acrylic acids are also present in waste streams and bottoms streams from acrylic acid purification processes, as well as waste and bottoms streams from ester production processes that use acrylic acid in the formation of the esters, such as, for example, butyl acrylate production.

Dimer crackers are typically used to recover acrylic acid value from the Michael addition products present in these streams.

Attempts have been made to improve acrylic acid recovery in dimer crackers.

U.S. Patent Application Publication No. 2004/0220427 discloses a process in which a dimer cracker operates at a high temperature and short residence time.

U.S. Pat. No. 7,569,721 discloses a process for conducting equilibrium-limited reactions using two reaction zones. The first reaction zone retains a least a portion of the product in a liquid phase, and at least a portion of the liquid from the first reaction zone is introduced into a second reaction zone to crack heavies and to vaporize at least a portion of the product.

U.S. Pat. No. 6,252,110 discloses a process for recovering acrylic acid from high boiling impurities by introducing the high boiling impurities into an acrylic acid recovery column, distilling acrylic acid from the column top and introducing bottom liquids from the acrylic acid recovery column into a pyrolyzing tank.

U.S. Pat. No. 6,482,981 discloses the use of a crystallization step to melt crystallize the acrylic acid and separate the acrylic acid into purified acrylic acid and a residual mother liquid, which is fed to an acrylic acid dimer decomposition step.

There is a need in the art for improved recovery of acrylic acid from Michael addition products.

### SUMMARY OF THE INVENTION

The invention is directed to a process for recovering acrylic acid from Michael addition products comprising:
a) feeding a stream comprising acrylic acid to a dehydration tower to produce an overhead stream comprising noncondensibles and lights and a bottoms stream comprising acrylic acid and Michael addition products of acrylic acid;
b) feeding at least a portion of the bottoms stream from the dehydration tower to a finishing tower to distill the bottoms stream of the dehydration tower to produce an acrylic acid-rich stream, and a bottoms stream comprising acrylic acid and Michael addition products of acrylic acid; and
c) optionally feeding acrylic acid from the acrylic acid-rich stream to an esterification process and/or a purification process, wherein:
   i) the esterification process produces an acrylic acid ester in an from the acrylic acid-rich stream and wherein a waste stream from the esterification process comprises Michael addition products of acrylic acid; and
   ii) the purification process produces a purified acrylic acid stream from the acrylic acid-rich stream and wherein a waste stream from the purification process comprises Michael addition products of acrylic acid;
d) feeding at least a portion of the bottoms stream from the finishing tower and/or the waste stream from the esterification process and/or purification process to a distillation column;
e) removing a distillate comprising acrylic acid and a bottoms stream comprising the Michael addition products of acrylic acid from the distillation column;
f) feeding at least a portion of the bottom from the distillation column to a dimer cracker reactor; and
g) recovering acrylic acid from a vapor stream exiting the dimer cracker reactor.

### BRIEF DESCRIPTION OF THE DRAWINGS

FIG. 1 is a schematic diagram showing the process according to an embodiment of the present invention.

FIG. 2 is a graph of the viscosity of the bottoms stream as a function of the percentage of trimers and higher-mers in the bottoms stream.

### DETAILED DESCRIPTION OF THE INVENTION

All percentage compositions are weight percentages (wt %), and all temperatures are in ° C., unless otherwise indicated. Averages are arithmetic averages unless otherwise indicated. An “average concentration” is the arithmetic average of the concentration entering a region and the concentration exiting the region, where the region is an individual reactor, a reactor system, or a zone within a reactor or reactor system. An “average ratio” is the ratio of the average concentration of one component relative to the average concentration of another component.

As used herein, the terms “Michael addition products of acrylic acid” and “dimer, trimer, and higher-mers of acrylic acid” refer to oligomers of acrylic acid. The oligomers of acrylic acid can reversibly react to form higher or lower oligomers, as well as acrylic acid.

The present invention relates to a process for producing acrylic acid and recovering acrylic acid from heavy waste byproducts, such as Michael addition products of acrylic acid.

In the present invention, acrylic acid is produced by any known acrylic acid production process. For example, acrylic acid can be made via propylene oxidation, a bio-based process (e.g., using any one of glycerol, 3-hydroxypropionic acid, lactate, lactic acid, etc., as a starting material), an ethylene cyanohydrin process, an acetylene process (i.e., the Reppe process), a β-propiolactone process, an acrylonitrile hydrolysis process, or a process performed by coupling carbon dioxide with ethylene or ethane. The reaction mixture produced during the acrylic acid production process may be processed in one or more unit operations. These operations may include, for example, cooling processes, quenching processes, separations, purifications, etc.

Preferably, the acrylic acid is produced by an exothermic, vapor stage, two-step propylene oxidation process. In a first step, propylene is oxidized in the presence of air and a first mixed metal oxide catalyst to produce acrolein. In a second step, the acrolein is oxidized in the presence of air and a second mixed metal oxide catalyst to produce a gaseous reaction mixture comprising acrylic acid.

The gaseous reaction mixture is cooled or quenched and partially condensed to form a liquid crude acrylic acid stream. The gaseous reaction mixture typically is superheated as it comes from the two-step oxidation process, wherein it contains more heat (energy) than the amount of heat required to vaporize the condensable components of the mixture. Preferably, the cooling step removes essentially all of the amount of superheat from the condensable portion of the gaseous reaction mixture. The cooling step can be conducted directly or indirectly in one or more pieces of equipment. For example, the cooling of the gaseous reaction mixture stream can be initiated in a quench or flash vessel, or can be integrated into the bottom of the dehydration column, with or without column internals. Preferably, the cooling step is initiated or primarily conducted in the dehydration column. The cooling step advantageously is initiated by bringing the gaseous reaction mixture stream into direct contact with a liquid, which preferably has a lower temperature. In one embodiment of the invention, the gaseous reaction mixture is introduced directly into the dehydration column where it is contacted with a liquid having a temperature lower than the temperature of the gaseous reaction mixture in order to at least partially cool the gaseous reaction mixture. In a preferred embodiment of the invention, the gaseous reaction mixture is introduced into the dehydration column and contacted with a spray of a cooler liquid.

Advantageously, the gaseous reaction mixture stream is cooled to a temperature of from about 50 to about 300° C., preferably from about 70 to about 200° C., below the boiling point of the majority of the highest-boiling components of the mixture. Preferably, the reaction gas mixture is cooled to a temperature of from about 50 to about 200° C., more preferably from about 60 to about 180° C.

The at least partially cooled gaseous reaction mixture stream is dehydrated. The dehydration preferably is conducted in the dehydration column, also referred to as the dehydration tower. The dehydration tower functions to remove the majority of water from the crude acrylic acid stream exiting as the dehydration tower bottoms stream. Advantageously, the dehydration tower is operated such that there is at least a bottoms stream and an overhead stream, and in some cases a side stream. Preferably, at least a portion of the overhead stream is condensed and is returned as a reflux liquid to the dehydration tower. Preferably a portion of the bottoms stream is cooled and used to cool the gaseous reaction mixture.

Preferably, essentially all noncondensibles and lights exit the dehydration tower in the overhead stream. Examples of noncondensibles present during the production of acrylic acid include, for example, nitrogen, oxygen, CO, carbon dioxide, and unreacted hydrocarbons such as propane and propylene. Advantageously, the overhead stream is introduced into a condenser, and at least a portion of the lights and acrylic acid are condensed and returned to the dehydration tower as a reflux stream.

The dehydration tower functions, at least partially, as a distillation column. However, as noted above, the dehydration column can also serve as a contacting zone for cooling of the gaseous reaction mixture. Preferably, the temperature of the bottoms stream from the dehydration tower is less than about 120° C. The temperature of the overhead stream from the dehydration tower is at least about 40° C.

The vent stream from the overhead condenser on the dehydration tower may be partially recycled to the reactor system. The remaining portion of the vent stream is removed from the separation system as a purge stream.

The bottoms stream, or liquid crude acrylic acid stream, from the dehydration tower advantageously is sent to a second column, except that a portion of this stream can be employed to cool the gaseous reaction mixture. In one embodiment of the invention, a portion of the bottoms stream from the dehydration tower is sent to a heat exchanger, which can be a reboiler. However, it is noted that the process can also be operated under conditions such that the heat exchanger is a cooler, depending on whether the process design requires heating or cooling. Preferably, a portion of the bottoms stream from the dehydration tower is fed to a second column. Advantageously, the feed point is near the top of the second column. The second column preferably is a distillation column and is used in conjunction with a reboiler and a condenser.

Preferably, the overhead stream from the second column is sent to a condenser. Preferably, the condenser is operated as a “total condenser” in that essentially all of the overhead stream is condensed. However, it is possible to remove a purge stream of noncondensible compounds from this condenser. A portion of the condensate of the second column is used to provide reflux and the remaining portion of the condensate is returned to the dehydration tower for lights removal. Advantageously, the condensate from the second column condenser is used to cool the gaseous reaction mixture, either as is or after additional heat exchange.

The bottoms stream from the second column advantageously is at least partially sent to the second column reboiler. The remainder of the bottoms stream can be incinerated or can be further treated to recover acrylic values. Preferably, the majority of acrylic acid is recovered from an acrylic acid-rich stream, such as, for example, a side draw stream, from the second column. A portion of the acrylic acid will be present in the bottoms stream of the second column. For example, the acrylic acid-rich stream may comprise technical grade acrylic acid (TGAA) and the bottoms stream may comprise primarily heavies, including Michael addition products of acrylic acid, soluble polymer and polymerization inhibitor residues. Alternatively, the acrylic acid-rich stream may comprise TGAA and the bottoms stream may comprise a lower grade stream of acrylic acid, such as ester grade acrylic acid (EGAA). Preferably, the acrylic acid-rich stream comprises at least 95 wt %, more preferably at least 96 wt %, even more preferably at least 97 wt %, and still more preferably at least 98 wt % acrylic acid based on the total weight of the acrylic acid-rich stream.

The process of the invention can remove the acrylic acid-rich stream at a point above or below the point where the liquid crude acrylic acid stream is fed to the second column, depending on the purity desired, i.e. the acrylic acid-rich stream can be removed above or below the feed in the second column. The acrylic acid-rich stream may be removed as a liquid or vapor.

The temperature and pressure in the second column can be determined according to design considerations well-known to those skilled in the art. Preferably, the second column is operated pressures so that the bottoms temperature is less than 120° C. More preferably, the second column is operated at pressures so that the bottoms temperature is less than 100° C. The advantage of allowing the second column to operate at lower temperatures is to minimize undesired dimer, oligomer and/or polymer formation. Advantageously, the temperature of the overhead stream as it leaves the second column is from about 40 to about 90° C. when producing acrylic acid and operating the second column at a head pressure of from about 40 to about 500 mm Hg. The temperature of the bottoms stream from the second column advantageously is from about 60 to about 100° C. when producing acrylic acid.

The design details of the dehydration column and of the second column, including their operating conditions such as temperatures, pressures, flow rates, equipment sizing including column height and diameters, choice of materials of construction, arrangement and choice of type of auxiliary equipment such as heat exchangers and pumps, choice and arrangement of column internals, and location of piping including take-off streams, can readily be determined by those skilled in the art according to well-known design considerations. Examples of distillation column configurations that can be used in the process of the invention include, for example, packed columns, trayed columns, divided wall columns, multi-stage devolatilizers, and the like. Any type of tray can be employed, including bubble trays, valve trays, cross flow trays, dual flow trays, and combinations thereof for those skilled in the art. Similarly, if packing is employed, any type of packing can be used, including randomly- or regularly-spaced packing. In a preferred embodiment of the invention, the dehydration column comprises packing in its upper section, and in its lower section provides for direct cooling of the incoming gaseous reaction mixture. Surge tanks optionally can be employed within the separation system such as, for example, at one or more locations between the dehydration column and the second column.

Steel alloys such as 316 SS and other higher alloys may be used as the material(s) of construction for the process equipment by using criteria well known to those skilled in the art for process equipment to be used in the distillation of corrosive streams. Alternatively, the process equipment can be partially constructed using copper or copper-containing alloys, such as various alloys sold under the name Monel. While not wishing to be bound by any theory, it is the believed that the presence of copper inhibits the undesired polymerization of acrylic acid.

The use of inhibitors is preferred in the process of the invention, regardless of the choice of materials of construction. Various compounds are well known to inhibit the reaction of acrylic acid, and are commercially available. Examples of preferred inhibitors include complexes and salts of manganese compounds, copper complexes and salts thereof, hydroquinone (HQ), monomethyl ether hydroquinone (MEHQ), phenothiazine (PTZ), 2,2,6,6-tetramethyl-1-piperidinyloxy (TEMPO), its derivatives, and related compounds such as 4-hydroxy TEMPO. Combinations of inhibitors can be employed that offer synergistic interactions and provide more effective polymerization inhibition than the additive performance of the individual inhibitors. In a preferred embodiment of the invention, a mixture of a source of soluble manganese ions, such as manganese acetate, and 4-hydroxy TEMPO are employed in the dehydration column as an inhibitor. Hydroquinone can also be added to this inhibitor mixture. It is also preferred, as is well-known in the art, to employ a molecular oxygen or air to the second column, as oxygen is known to be a polymerization retarder. The inhibitor is employed in an amount sufficient to prevent or reduce the polymerization of acrylic acid, as is well known to those skilled in the art.

Preferably, the dehydration tower and second column form a coupled distillation tower system. The coupled distillation tower system comprises the dehydration tower and a finishing tower (i.e., the second column). Other suitable process configurations are possible and are known to those skilled in the art which use intermediate tanks to contain certain process streams prior to being fed to each tower.

Preferably, at least a portion of the acrylic acid in the acrylic acid-rich stream is fed to an esterification process and/or to a purification process. For example, the acrylic acid-rich stream may be split and a portion of the acrylic acid is sent to the esterification process and the remainder of the acrylic acid is sent to a purification process.

In the esterification process, acrylic acid from the acrylic acid-rich stream is converted to an ester of acrylic acid. Preferably, the acrylic acid ester formed in the esterification process is selected from the group consisting of methyl acrylate, ethyl acrylate, butyl acrylate, and 2-ethyl hexyl acrylate. More preferably, the acrylic acid ester formed in the esterification process is butyl acrylate. The esterification process may comprise one or more unit operations to purify the acrylic acid ester to produce a product stream and a waste stream comprising heavies including Michael addition products of acrylic acid.

In the purification process, acrylic acid from the acrylic acid-rich stream is purified to form a purified acrylic acid stream. The purification process may comprises one or more unit operations, such as distillation or crystallization, to remove undesired byproducts or contaminants present in the acrylic acid-rich stream to produce a purified acrylic acid stream. The purification process also produces a waste stream comprising heavies including Michael addition products of acrylic acid.

The bottoms stream of the second column and/or the waste stream from the esterification process and/or purification process is fed to a distillation column for the recovery of acrylic values in the bottoms stream, referred to herein as “the recovery column.” Preferably, the waste stream from the esterification process and/or the purification process is fed to the recovery column. The bottoms stream of the second column and the waste stream from the esterification process and/or purification process comprise acrylic acid and Michael addition products of acrylic acid. Preferably, AOPA is the majority component of the Michael addition products. More preferably, AOPA is the majority component in the bottoms stream of the second column and the waste stream from the esterification process and/or the purification process. In the inventive process, acrylic acid recovery in a dimer cracker is improved by driving the reaction towards the formation of acrylic acid by removing acrylic acid from the bottoms stream and/or waste stream in the recovery column before the bottoms stream and/or waste stream enters the dimer cracker. By removing the acrylic acid in the recovery column, the reaction will drive the formation of acrylic acid from 3-(acryoloyloxy)propanoic acid (AOPA) to acrylic acid to a greater degree than would have occurred, thus increasing the recovery of AOPA as acrylic acid. This will in turn drive the reaction of the trimer to AOPA, as well as the cracking of any higher-mers present in the dimer cracker to lower oligomers.

Preferably, the bottoms stream and/or waste stream fed to the recovery column comprises less than 5 wt % of water based on the total weight of the liquid stream. More preferably, the bottoms stream comprises less than 3 wt %, even more preferably less than 2 wt %, yet more preferably less than 1 wt %, and still more preferably less than 0.5 wt % of water based on the total weight of the bottoms stream and/or waste stream.

The recovery column comprises 0.1 to 30 theoretical stages of separation, preferably from 1 to 10 theoretical stages of separation. The recovery column may contain baffles in at least a portion of the recovery column.

The distillate may be condensed in a condenser to form a liquid overhead product stream comprising acrylic acid. Preferably, at least a portion of the overhead product stream is recycled to the recovery column as reflux. Preferably the reflux ratio is at least 0.1, more preferably at least 0.5.

The recovery column is preferably maintained at a temperature of at least 150° C. More preferably, the recovery column is maintained at a temperature of at least 165° C.

The bottoms stream of the recovery column comprises 5 to 95 wt % of AOPA and other acrylic acid Michael addition products, including trimers and tetramers of acrylic acid based on the total weight of the bottoms stream. Preferably, at least 50 wt % of the Michael addition products of acrylic acid in the bottoms stream comprise AOPA. Preferably, the bottoms stream of the recovery column comprises 20 to 95 wt % of AOPA and other acrylic acid Michael addition products, more preferable 40 to 95 wt % of AOPA and other acrylic acid Michael addition products, and even more preferably 60 to 95 wt % of AOPA and other acrylic acid Michael addition products based on the total weight of the bottoms stream of the recovery column.

A polymerization inhibitor may be added to the recovery column along with the liquid stream. Alternatively, the recovery column may contain polymerization added previously in the process.

The bottoms stream of the recovery column comprising the Michael addition products of acrylic acid are fed to a dimer cracker reactor, where the Michael addition products are cracked to form acrylic acid and lower oligomer Michael addition products. For example, tetramer can be cracked to form trimer, and trimer can be cracked to form AOPA, while AOPA is cracked to form acrylic acid. Because a large percentage of the acrylic acid is removed as a distillate in the recovery column, the reversible reaction of acrylic acid and Michael addition products favors the production of acrylic acid and lower oligomers of Michael addition products in the dimer cracker compared to a dimer cracker in which the percentage by weight of acrylic acid entering the dimer cracker reactor is higher.

The dimer cracker reactor may comprise a single vessel or more than one vessel. For example, the dimer cracker reactor may comprise a continuously stirred tank reactor, or a continuously stirred tank reactor followed by a plug flow reactor.

The dimer cracker reactor may be catalyzed or uncatalyzed. Preferably, the dimer cracker reactor is uncatalyzed.

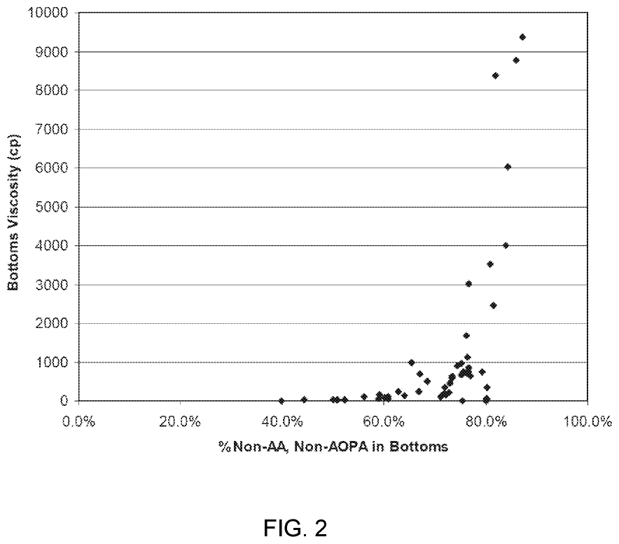

As shown in FIG. 2, the viscosity of the bottoms stream can vary greatly based on the amount of non-acrylic acid and non-AOPA in the bottoms stream. The viscosity of the bottoms stream increases significantly when the amount of trimer and tetramer Michael addition products increases.

By driving the reversible reaction to favor formation of acrylic acid and lower oligomers, it may be easier to control the viscosity of the dimer cracker reactor. As trimer is reduced to acrylic acid and AOPA, the viscosity of the reactor mixture may be maintained at an operable level.

In the recovery column, the viscosity of the bottoms stream can be controlled by adjusting the pressure of the recovery column. Alternatively, the viscosity may also be adjusted with temperature or by adding a lower-viscosity material, such as an oil.

Preferably, the dimer cracker reactor is maintained at a temperature of at least 150° C., more preferably at least 165° C.

Acrylic acid is recovered in the vapor stream of the dimer cracker reactor. Preferably, the recovered acrylic acid is returned to the recovery column.

The residence time in the recovery column and dimer cracker reactor is preferably less than 2 hour, more preferably less than 0.5 hour.

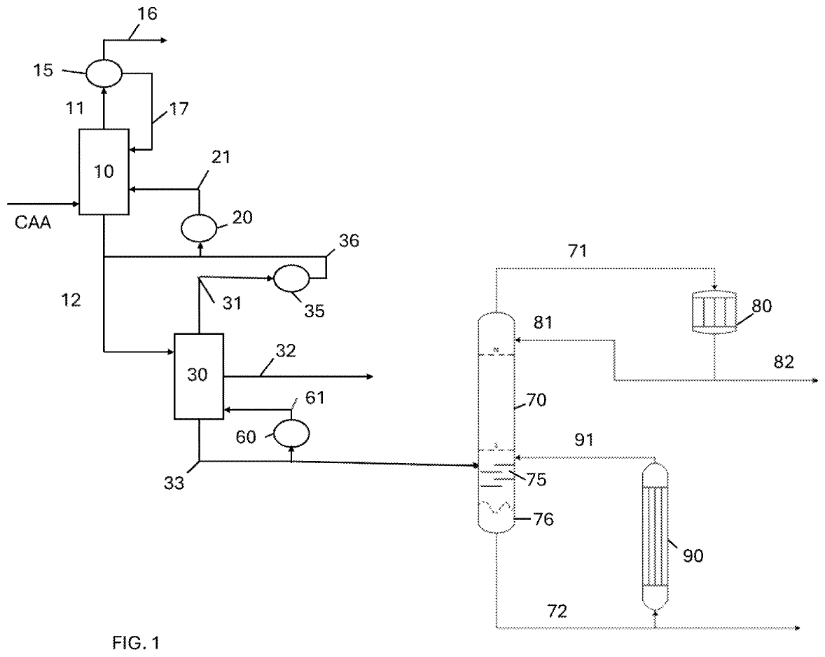

One embodiment of the present invention is shown in FIG. 1. A crude acrylic acid stream CAA is fed to a dehydration tower 10, where the gaseous crude acrylic acid stream is quenched and condensed. The lights 11 from dehydration tower 10 are sent to condenser 15. Condensible materials 17 are returned to dehydration tower 10 as reflux. Lights and noncondensible materials 16 may be vented or incinerated. A portion of the bottoms stream 12 is sent to a heat exchanger 20 and liquid stream 21 exiting the heat exchanger 20 is returned to dehydration tower 10 as a cooling liquid.

The remainder of bottoms stream 12 is fed to second column 30, which functions as a distillation column. An overhead stream 31 from second column 30 is condensed in condenser 35 and the condensed overhead stream 36 is returned to dehydration tower 10. A side draw 32 comprising primarily acrylic acid is removed from second column 30. A bottoms stream 33 comprising acrylic acid and heavies exits the bottom of finishing column 30. A portion of bottoms stream 33 may be sent to reboiler 60 for recycling to the finishing column 30 as stream 61.

At least a portion of bottoms stream 33 is fed into distillation column 70 comprising N trays (N<30) and baffles 75. Distillate 71 is condensed in condenser 80. A portion of the condensed distillate 81 is returned to distillation column 70 as reflux, and the remainder is drawn off as product stream 82. The bottoms collect in distillation sump 76 and bottoms stream 72 containing the Michael addition products exits the bottom of the distillation column 70. Bottoms stream 72 is fed to dimer cracker reactor 90 to convert AOPA to acrylic acid and higher oligomers to acrylic acid and AOPA. The vapor stream 91 exiting dimer cracker 90 is returned to distillation column 70.

### EXAMPLES

### Example 1

The experimental equipment used for Examples 1 and 2 comprised a reactor which is also a reboiler for the distillation column connected to a 1.5 inch oldershaw distillation column containing 10 trays. The vapors from the distillation column were sent to a condenser and part of the condensate obtained was refluxed back to the top tray of the column while the remainder was collected as distillate. Both the condenser and the top tray were supplied with inhibitor. The distillation column and sump were maintained under vacuum at the desired operating pressure using a vacuum pump. The pressure in the reactor/reboiler was maintained at a higher pressure than the distillation column, 5-10 psig.

The volume of the reactor/reboiler and the distillation sump were approximately equal. The temperature was measured at the reactor outlet and in the distillation sump. The feeds were introduced in the system upstream of the reactor/reboiler and downstream of the bottoms product takeoff location. The reactor/reboiler was maintained at pressure by means of a back pressure regulator. An in-line viscometer was in the reactor/reboiler recirculation loop to record the viscosity of the effluent at the sump temperature.

The experiment was conducted at a condenser pressure of 215 mmHg. The feed comprised 36.4% acrylic acid, 32.9% acrylic acid dimer, 7.5% acrylic acid trimer, 0.1301% maleic acid and 2.1% hydroquinone. An inhibitor was added to the overheads to prevent polymerization. The results are shown below in Table 1.

Dimer recovery is calculated using Eq. 1 below:

$$Dimer Recovery ( \% ) = \frac{{n}_{A A , D} - ( {n}_{AA , F} + {n}_{AA , I} )}{2 \times ( {n}_{A O PA , F} )} \qquad Eq . 1$$

$$where ,$$

$${n}_{i , j} = molar flow rate of component ‘ i ’ in material stream ‘ j ’ .$$

TABLE 1

| | Reactor | Distillation | | Rxn Residence | | | AOPA | Percent | Bottoms |
|---|---|---|---|---|---|---|---|---|---|
| Example | Outlet | Sump Temp | Pressure | Time based | OH/Bot | Reflux | Recovery | AA in | Viscosity |
| # | Temp (C.) | (C.) | (mmHg) | on feed (hr) | Split | Ratio | (%) | OH (wt %) | Sump T (cp) |
| 1 | 180 | 171 | 215 | 0.44 | 2.7 | 1 | 106% | 99% | 40 |
| 2 | 177 | 171 | 294 | 0.42 | 1.8 | 1 | 77% | 99% | 7.3 |
| 3 | 175 | na | 150 | 1.02 | 2.4 | 0 | 108% | 97% | 85* |
| 4 | 175 | na | 50 | 0.44 | 2.1 | 0 | 80% | 77% | 20* |

*Viscosity measured at 165 C.

### Example 2

Example 2 in the table above was conducted in the same equipment as example 1, but at a condenser pressure of 294 mmHg. The feed comprised of 36.4% acrylic acid, 32.9% acrylic acid dimer, 7.5% acrylic acid trimer, 0.1301% maleic acid and 2.1% hydroquinone. An inhibitor was added to the overheads to prevent polymerization.

### Comparative Example 3

The experimental equipment used for Comparative Examples 3 and 4 was a CSTR in which lights such as acrylic acid were obtained as the overhead product and heavier products such as higher oligomers of acrylic acid were bottoms product.

In comparative example 3, the CSTR was fed with 26.69% acrylic acid, 36.67% acrylic acid dimer, 7.46% acrylic acid trimer. The CSTR was maintained at an operating pressure of 150 mmHg. None of this overhead stream was refluxed back to the CSTR.

### Comparative Example 4

In comparative example 4, the equipment was identical to that of Comparative Example 3. The CSTR was fed with 27.48% acrylic acid, 31.25% acrylic acid dimer, 6.65% acrylic acid trimer. The CSTR was maintained at an operating pressure of 50 mmHg. None of this overhead stream was refluxed back to the CSTR.

1. A process for recovering acrylic acid from Michael addition products comprising:
a) feeding a stream comprising acrylic acid to a dehydration tower to produce an overhead stream comprising noncondensibles and lights and a bottoms stream comprising acrylic acid and Michael addition products of acrylic acid;
b) feeding at least a portion of the bottoms stream from the dehydration tower to a finishing tower to distill the bottoms stream of the dehydration tower to produce an acrylic acid-rich stream comprising acrylic acid, and a bottoms stream comprising acrylic acid and Michael addition products of acrylic acid;
c) optionally feeding acrylic acid from the acrylic acid-rich stream to an esterification process and/or a purification process, wherein:
i) the esterification process produces an acrylic acid ester in an from the acrylic acid-rich stream and wherein a waste stream from the esterification process comprises Michael addition products of acrylic acid; and
ii) the purification process produces a purified acrylic acid stream from the acrylic acid-rich stream and wherein a waste stream from the purification process comprises Michael addition products of acrylic acid;
d) feeding at least a portion of the bottoms stream from the finishing tower and/or the waste stream from the esterification process and/or the purification process to a distillation column;
e) removing a distillate comprising acrylic acid and a bottoms stream comprising the Michael addition products of acrylic acid from the distillation column;
f) feeding at least a portion of the bottom from the distillation column to a dimer cracker reactor; and
g) recovering acrylic acid from a vapor stream exiting the dimer cracker reactor.

2. The process of claim 1, wherein step d) comprises feeding at least a portion of the waste stream from the esterification process and/or the purification process to the distillation column.

3. The process of claim 1, wherein the bottoms stream of the distillation column comprises 5 to 95 wt % 3-(acryoloyloxy) propanoic acid and Michael addition products of acrylic acid based on the total weight of the bottoms stream.

4. The process of claim 1, wherein the distillation column comprises 0.1 to 30 theoretical stages of separation.

5. The process of claim 4, wherein the distillation column comprises 1 to 10 theoretical stages.

6. The process of claim 1, further comprising controlling the viscosity of the bottoms stream of the distillation column by adjusting the pressure of the distillation column.

7. The process of claim 1, wherein a polymerization inhibitor is fed to the distillation column.

8. The process of claim 1, wherein the distillation column and the dimer cracker are maintained at a temperature greater than 150° C.

9. The process of claim 8, wherein the distillation column and the dimer cracker are maintained at a temperature greater than 165° C.

10. The process of claim 1, wherein condensed reflux from the distillate of the distillation column is recycled to the distillation column at a reflux ratio of at least 0.1.

11. The process of claim 10, wherein condensed reflux from the distillate of the distillation column is recycled to the distillation column at a reflux ratio of at least 0.5.

12. The process of claim 1, wherein the dimer cracker comprises a continuously stirred tank reactor.

13. The process of claim 1, wherein the liquid stream comprises 5 wt % water based on the total amount of the liquid stream entering the distillation column

14. The process of claim 1, wherein the acrylic acid in the stream comprising acrylic acid fed to the dehydration tower is produced by a method selected from the group consisting of propylene oxidation-based process, a bio-based process, an ethylene cyanohydrin-based process, an acetylene-based process, a β-propiolactone-based process, an acrylonitrile hydrolysis-based process, or a process performed by coupling carbon dioxide with ethylene or ethane.

15. The process of claim 14, wherein the acrylic acid in the stream fed to the dehydration tower is produced by an exothermic vapor phase propylene oxidation-based process.
